(19) United States
(12) Patent Application Publication
(10) Pub. No.: US 2020/0318058 A1
(43) Pub. Date: Oct. 8, 2020

(54) OBSERVATION SYSTEM AND OBSERVATION METHOD USING THE SAME

(71) Applicant: OLYMPUS CORPORATION, Tokyo (JP)

(72) Inventors: Tsuyoshi Mochizuki, Musashino-shi (JP); Koh Mohri, Hachioji-shi (JP); Yoshihito Iguchi, Hino-shi (JP); Shogo Usui, Machida-shi (JP); Shintaro Takahashi, Hachioji-shi (JP); Asuka Nakamura, Sagamihara-shi (JP); Shoichi Kaneko, Hachioji-shi (JP); Ikutoshi Fukushima, Fuchu-shi (JP); Masaru Mizunaka, Hino-shi (JP)

(73) Assignee: OLYMPUS CORPORATION, Tokyo (JP)

(21) Appl. No.: 16/904,799

(22) Filed: Jun. 18, 2020

Related U.S. Application Data
(63) Continuation of application No. PCT/JP2018/046825, filed on Dec. 19, 2018.

(30) Foreign Application Priority Data
Dec. 19, 2017 (JP) 2017-242716

(51) Int. Cl. C12M 1/34; G01N 21/25; G01N 21/31; C12M 1/00; C12M 1/36; G06T 7/90; G06T 7/00; H04N 5/225

(52) U.S. Cl. CPC C12M 41/26; G01N 21/255; G01N 21/31; C12M 31/00; C12M 41/36; C12M 41/48; G06T 2207/10024; G06T 7/90; G06T 7/0012; H04N 5/2256; G06T 2207/10152; G06T 2207/30024; G06T 2207/30242; C12M 23/22

(57) ABSTRACT

An observation system includes an illumination unit including a light source from which emits an illumination light including light of a plurality of colors, and light receiving unit including an optical sensor which acquires a light intensity. The illumination unit emits the illumination light for a culture vessel so that the illumination light travels from a first side to a second side of the culture vessel, the first side and the second side being defined by interposing a culture medium. The light receiving unit receives, on the first side, light emitted from the illumination unit, entering the culture vessel from the first side, reflected at the second side, and transmitted through the culture medium, and acquires a light intensity for each color in the received light for calculation of a pH value of the culture medium.

### CROSS-REFERENCE TO RELATED APPLICATION

This application is a Continuation Application of PCT Application No. PCT/JP2018/046825, filed Dec. 19, 2018 and based upon and claiming the benefit of priority from the prior Japanese Patent Application No. 2017-242716, filed Dec. 19, 2017, the entire contents of which are incorporated herein by reference.

### BACKGROUND

The present invention relates generally to an observation system and an observation method using the observation system.

There is a demand to understand the state of cultured cells etc. in culture, when the cells etc. are cultured in an incubator. Thus, generally, a culture vessel is suitably taken out of the incubator and the cultured cells etc. are observed with a microscope. In addition, the pH of the culture medium is also of interest with regards to a state of the cultured cells. For example, US 2012/0214250 A1 discloses a pH measuring device capable of measuring the pH of the culture medium during cell culturing, and a special culture vessel for the measuring device.

### SUMMARY

According to an exemplary embodiment, an observation system includes an illumination unit including a light source from which emits an illumination light including light of a plurality of colors, and configured to emit the illumination light for a culture vessel so that the illumination light travels from a first side to a second side of the culture vessel, the culture vessel being configured to store a culture medium for culturing a cell, and the first side and the second side being defined by interposing the culture medium, and a light receiving unit including an optical sensor which acquires a light intensity, and configured to receive, on the first side, light emitted from the illumination unit, entering the culture vessel from the first side, reflected at the second side, and transmitted through the culture medium, and to acquire a light intensity for each color in the received light, the light intensity being used for calculation of a pH value of the culture medium.

According to an exemplary embodiment, an observation method using an observation system including an illumination unit including a light source from which emits an illumination light including light of a plurality of colors, and configured to emit the illumination light for a culture vessel so that the illumination light travels from a first side to a second side of the culture vessel, the culture vessel being configured to store a culture medium for culturing a cell, and the first side and the second side being defined by interposing the culture medium and a light receiving unit including an optical sensor which acquires a light intensity, and configured to receive, on the first side, light emitted from the illumination unit, entering the culture vessel from the first side, reflected at the second side, and transmitted through the culture medium, and to acquire a light intensity for each color in the received light, the light intensity being used for calculation of a pH value of the culture medium, wherein the optical sensor includes an image sensor which acquires a light intensity and an image, the image sensor is configured to receive the light of the plurality of colors transmitted through the culture medium, and acquire an image the cell and the light intensity for each color in the received light for calculation of the pH value of the culture medium, includes acquiring an image of the cell by setting a focus position of the light receiving unit on the cell, acquiring an image of the culture medium by setting the focus position of the light receiving unit on the culture medium; and calculating a pH value of the culture medium based on the image of the culture medium.

According to an exemplary embodiment, a non-transitory computer-readable storage medium stores a program to control an observation system including an illumination unit including a light source from which emits an illumination light including light of a plurality of colors, and configured to emit the illumination light for a culture vessel so that the illumination light travels from a first side to a second side of the culture vessel, the culture vessel being configured to store a culture medium for culturing a cell, and the first side and the second side being defined by interposing the culture medium and a light receiving unit including an optical sensor which acquires a light intensity, and configured to receive, on the first side, light emitted from the illumination unit, entering the culture vessel from the first side, reflected at the second side, and transmitted through the culture medium, and to acquire a light intensity for each color in the received light, the light intensity being used for calculation of a pH value of the culture medium, wherein the optical sensor includes an image sensor which acquires a light intensity and an image, the image sensor is configured to receive the light of the plurality of colors transmitted through the culture medium, and acquire an image of the cell and the light intensity for each color in the received light for calculation of the pH value of the culture medium, and wherein the program causes a computer of the observation system to operate a process of acquiring an image of the cell by setting a focus position of the light receiving unit on the cell, acquire an image of the culture medium by setting the focus position of the light receiving unit on the culture medium, and calculate a pH value of the culture medium based on the image of the culture medium.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view of a configuration example of an observation system according to a first embodiment.

FIG. 2 is a block diagram schematically showing a configuration example of the observation system according to the first embodiment.

FIG. 3 is a schematic view showing a configuration example of an image acquisition unit and a sample according to the first embodiment.

FIG. 4 is a schematic view showing a configuration example of an illumination unit and an imaging optical system according to the first embodiment, and is given as a plane including an optical axis of an objective lens.

FIG. 5 is a schematic view showing a configuration example of the illumination unit and the imaging optical system according to the first embodiment, and is given as a plane perpendicular to the optical axis of the objective lens.

FIG. 6 is a block diagram schematically showing exemplary functions of the observation system according to the first embodiment.

FIG. 7 is a flowchart schematically showing an example of an operation of the observation system according to the first embodiment.

FIG. 8 is a schematic view showing a configuration example of an observation system according to a first modification of the first embodiment.

FIG. 9 is a schematic diagram of a configuration example of an observation system according to a second embodiment.

### DETAILED DESCRIPTION

### First Embodiment

Hereinafter, a first embodiment will be described with reference to the accompanying drawings. An observation system according to the present embodiment is a system for imaging cells, cell groups, and tissues etc. in culture, and recording and analyzing the number and morphology etc. of the cells or cell groups. The observation system has a function of measuring the pH (hydrogen ion index) value of a culture solution based on the color of the culture solution. The observation system has a function of calculating the proliferation capability of cells etc. on the basis of the number of cells and a change in pH value.

### Configuration of Observation System

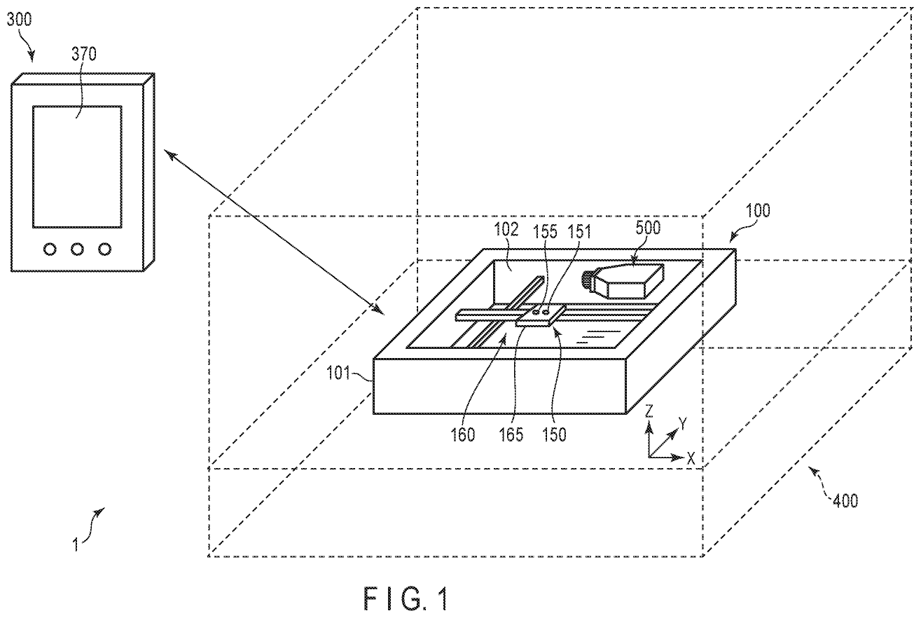

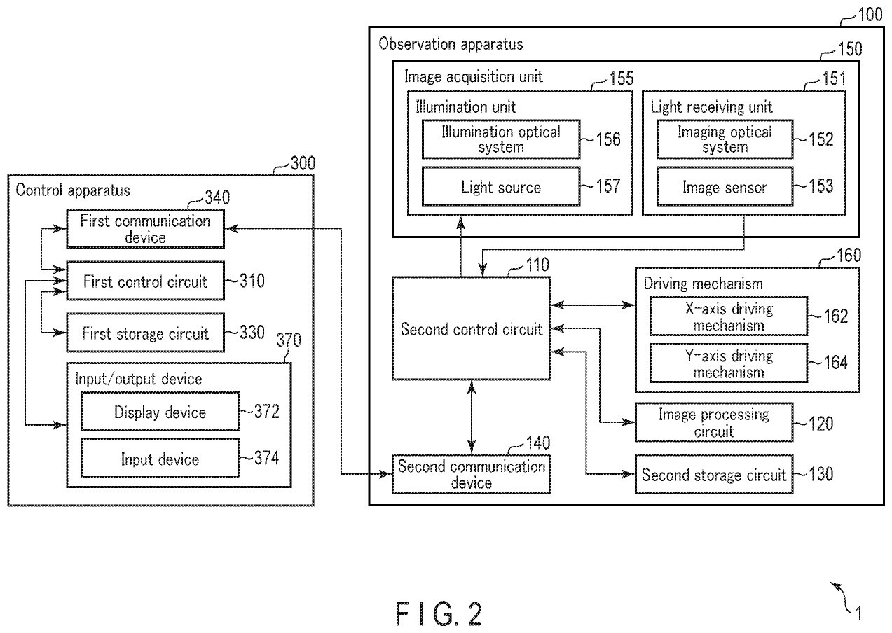

FIG. 1 is a schematic diagram showing a general appearance of the observation system 1. FIG. 2 is a block diagram schematically showing a configuration example of the observation system 1. The observation system 1 includes an observation apparatus 100, and a control apparatus 300. The observation apparatus 100 has a substantially flat plate shape. A sample 500 to be observed is disposed on the upper surface of the observation apparatus 100. The observation apparatus 100 and the sample 500 are installed, for example, in an incubator 400. The incubator 400 is a general incubator for cell culture. The incubator 400 is, for example, a CO₂ incubator. For the following description, X and Y axes perpendicular to each other are defined in a plane parallel to the plane on which the sample 500 of the observation apparatus 100 is placed, and a Z axis is defined so as to be perpendicular to the X and Y axes.

The observation apparatus 100 includes a housing 101 and an image acquisition unit 150. A transparent plate 102 as a transparent member having an optically transparent characteristic is disposed on at least part of the upper surface of the housing 101. The image acquisition unit 150 is provided inside the housing 101. The image acquisition unit 150 includes a light receiving unit 151 having an image sensor 153, an illumination unit 155 having a light source 157, and a support unit 165. As shown in FIG. 1, the light receiving unit 151 and the illumination unit 155 are provided on the support unit 165. The light receiving unit 151 and the illumination unit 155 are provided close to each other. The illumination unit 155 illuminates the sample 500 via the transparent plate 102, and the light receiving unit 151 acquires an image of the sample 500 via the transparent plate 102.

The control apparatus 300 is installed, for example, outside the incubator 400. The control apparatus 300 communicates with the observation apparatus 100. The control apparatus 300 transmits various instructions to the observation apparatus 100, and acquires and analyzes data obtained from the observation apparatus 100.

The observation system 1 can image a wide range of the sample 500 by repeatedly imaging the sample 500 while moving the image acquisition unit 150 in the X-axis direction and the Y-axis direction during an observation operation. Further, the observation system 1 can repeatedly perform such an observation operation at intervals set according to a predetermined sequence.

### Sample

The sample 500 to be measured by the observation system 1 is, for example, as follows. The sample 500 includes, for example, a vessel including a culture medium in which cells are being cultured. The culture vessel can be, for example, a petri dish, a culture flask, a multiwell plate, or the like. As such, the shape, size and the like of the culture vessel are not limited. The culture vessel is made of an optically transparent material such as a resin. One of the measurement targets of the observation system 1 is, for example, a culture medium in which cells are being cultured. The culture medium is, for example, a liquid culture medium with phenol red added. The culture medium may be any other liquid or solid culture medium. Another measurement target of the observation system 1 is cells. The cells to be measured. are, for example, cultured cells, which may be adherent cells or floating cells. The cells may be spheroids or tissues. Further, the cell may be derived from any organism, and may be a bacterium or the like. As described above, the sample 500 includes a logical sample that is an organism or a sample derived from the organism.

### Control Apparatus

The control apparatus 300 controls the entire observation system 1. The control apparatus 300 is, for example, a personal computer (PC), a tablet-type information terminal, or the like. FIG. 1 shows a tablet-type information terminal.

Further, the control apparatus 300 is provided with an input/output device 370. As shown in FIG. 2, the input/output device 370 includes a display device 372 such as a liquid crystal display and an input device 374 such as a touch panel. The input device 374 may include a switch, a dial, a keyboard, a mouse, and the like, in addition to the touch panel.

Further, the control apparatus 300 is provided with a first communication device 340. The first communication device 340 is a device for communicating with the observation apparatus 100. Various wired communications may be used for this communication. Further, this communication may use a wireless communication standard such as Wi-Fi or Bluetooth. The control apparatus 300 and the observation apparatus 100 may each be connected to a communication network such as the Internet and communicate with each other via the network.

Further, the control apparatus 300 includes a first control circuit 310 and a first storage circuit 330. The first control circuit 310 controls the operation of each unit of the control apparatus 300. The first control circuit 310 performs various calculations related to the control for measuring the sample 500, controls the operation of the display device 372, controls the storing of information in the first storage circuit 330, and controls the communication with the observation apparatus 100 via the first communication device 340. In addition, the first control circuit 310 may perform various analyses based on the image acquired from the observation apparatus 100. For example, the first control circuit 310 may extract an image of a cell or a cell group included in the sample 500, calculate the number of cells or cell groups, specify the pH value of the culture medium, and calculate the proliferation capability based on the number of cells and the pH value.

The first storage circuit 330 stores, for example, programs and various parameters used in the first control circuit 310. The first storage circuit 330 stores data obtained by and received from the observation apparatus 100.

### Observation Apparatus

As shown in FIG. 1, the transparent plate 102 is disposed on at least part of the upper surface of the housing 101 of the observation apparatus 100. The transparent plate 102 is formed of a transparent member having an optically transparent characteristic, such as glass. The transparent plate 102 is provided so as to be horizontal when the observation apparatus 100 is installed. The sample 500 is left still on the transparent plate 102. As described above, the upper surface of the housing 101 is an arrangement surface for the sample 500 to be disposed. The housing 101 including the transparent plate 102 has a sealed structure, and the observation apparatus 100 has a closed internal space. Although the observation apparatus 100 is intended for installation in a high temperature and high humidity environment such as in the incubator, the environment inside the housing 101 can be suitably maintained for, for example, an electronic apparatus, an optical device, and so on.

The image acquisition unit 150 provided inside the housing 101 includes the light receiving unit 151 and the illumination unit 155. The light receiving unit 151 and the illumination unit 155 are fixed to the support unit 165, and move integrally as described later.

As shown in FIG. 2, the illumination unit 155 includes an illumination optical system 156 as well as the light source 157. The illumination light emitted from the light source 157 travels toward the transparent plate 102 through the illumination optical system 156, that is, toward the sample 500. The light source 157 includes, as a non-limiting example, a light emitting diode (LED).

The light receiving unit 151 includes an imaging optical system 152 as a light receiving optical system and the image sensor 153 as a light receiving device. The light receiving unit 151 generates image data based on an image formed on the imaging area of the image sensor 153 via the imaging optical system 152. The imaging optical system 152 has a focus lens, and can change a focus position in the Z-axis direction. Further, it is preferable that the imaging optical system 152 be a zoom optical system that can change a focal length. The light receiving unit 151 performs imaging in the direction of the transparent plate 102, that is, the direction of the sample 500, and acquires an image of the sample 500.

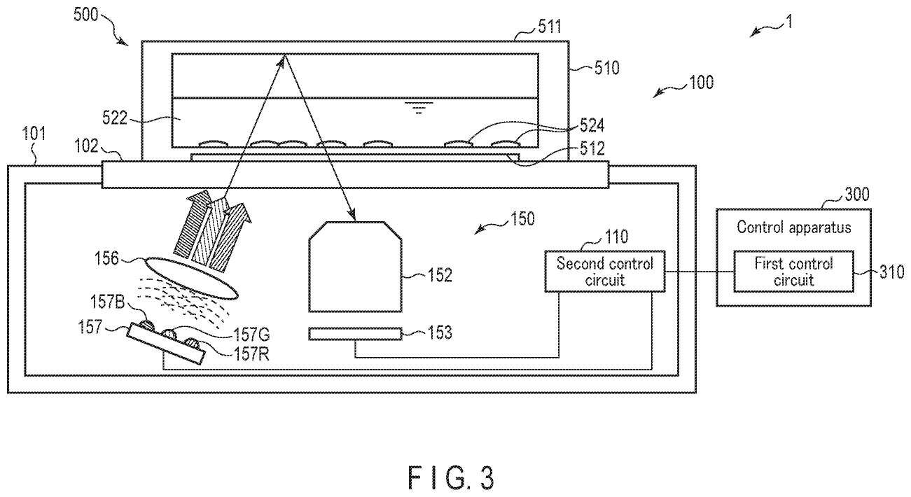

FIG. 3 is a schematic diagram showing a configuration example of the image acquisition unit 150 and the sample 500. FIG. 3 schematically shows a state of the sample 500 in which a liquid culture medium 522 is placed in a culture vessel 510 and cells 524 are cultured in the liquid culture medium 522. The Light source 157 includes a plurality of LEDs so that each of, for example, red light, green light, and blue light can be emitted individually. That is, the light source 157 includes a red light source 157R for emitting red light, a green light source 157G for emitting green light, and a blue light source 157B for emitting blue light. The red light source 157R, green light source 157G, and blue light source 157B may be provided in any number.

As shown in FIG. 3, the illumination light emitted from the light source 157 illuminates the sample 500 on the transparent plate 102 through the illumination optical system 156. The illumination light passes through a vessel bottom portion 512 of the culture vessel 510 and the culture medium 522 within the culture vessel 510, reaches a vessel upper portion 511 of the culture vessel 510, and is reflected by the vessel upper portion 511. The vessel upper portion 511 is an upper wall when the culture vessel 510 is a flask, and it is a lid when the culture vessel 510 is a petri dish. The reflected light illuminates the cells 524 and the like in the culture medium 522 and enters the imaging optical system 152. The light receiving unit 151 performs an imaging operation on the light incident on the imaging optical system 152.

The image sensor 153 is, for example, a monochromatic image sensor. The red light, green light, and blue light are sequentially emitted from the light source 157 in a time division manner, and the monochromatic image sensor 153 sequentially receives them and acquires images according to the color of the illumination light, whereby the observation apparatus 100 can acquire color images. That is, based on the images obtained here, the user can grasp the color of the sample 500. On the basis of these images, the transmittance for each color (for each wavelength) of the sample 500 can be calculated. For example, when the culture medium 522 in the sample 500 contains a dye whose color changes with pH, such as phenol red, the pH value can be calculated based on the light transmittance of each color (wavelength).

The imaging optical system 152 may be an optical system for a single wavelength in the same way that a monochromatic image sensor is used as the image sensor 153. Since the image sensor 153 and the imaging optical system 152 are for a single color, the image sensor 153 and the imaging optical system 152 are relatively inexpensive as compared with a color optical system in which chromatic aberration and the like are taken into consideration. Chromatic aberration does not affect the pH value measurement. When the shape and number of cells are obtained from an image, a color image is not often required. Therefore, an image having a sufficient amount of information can be obtained even if only monochromatic illumination is used in acquiring images. In this case, the imaging optical system 152 may be an optical system for a single wavelength. Further, with the configuration of acquiring images of the respective colors in a time division manner, it is also possible to acquire a color image with a good image quality by correcting the chromatic aberration in image processing when a color image is created. Of course, by using an optical system for detecting a color image as the imaging optical system 152, a high-quality color image with reduced chromatic aberration can be obtained regardless of image processing or the like.

Further, by configuring the imaging optical system 152 so that an appropriate filter can be inserted into and removed from it, the observation apparatus 100 can take a configuration capable of acquiring, for example, a fluorescence image.

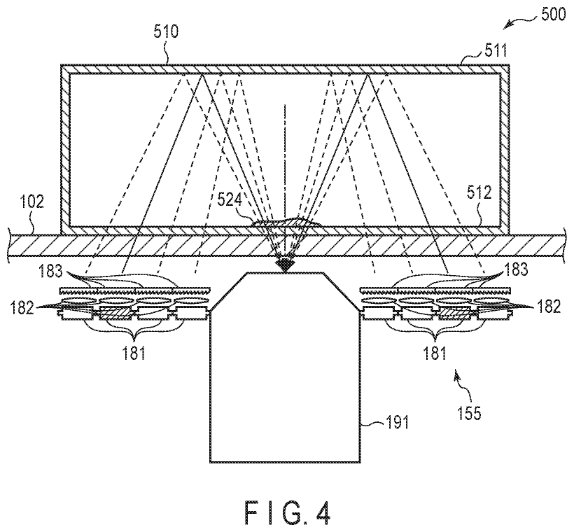

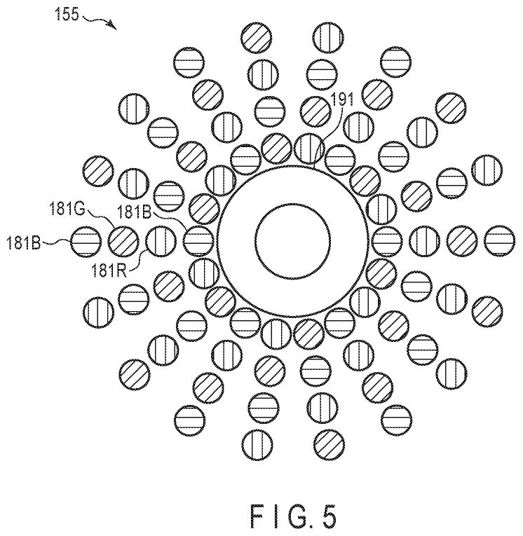

An example of the configuration of the illumination unit 155 and the imaging optical system 152 will be described with reference to FIGS. 4 and 5. FIG. 4 is a schematic diagram showing a plane including the optical axis of an objective lens 191 included in the imaging optical system 152. FIG. 5 is a schematic diagram showing a plane perpendicular to the optical axis of the objective lens 191.

As shown in FIG. 4, a sample 500 including cells 524 is disposed on the transparent plate 102. The illumination unit 15 includes a plurality of LED light sources 181 as the light source 157, arranged at intervals in the circumferential direction and the radial direction around the objective lens 191. As the illumination optical system 156, the illumination unit 155 is provided with a plurality of condenser lenses 182 and a plurality of diffusion plates 183. The condenser lenses 182 are arranged corresponding to the respective LED light sources 181 for condensing the illumination light generated in the respective LED light sources 181. The diffusion plates 163 diffuse the illumination light condensed by the condenser lenses 182.

The illumination unit 155 can independently turn on specific one or ones of the LIED light source 181. For example, in FIG. 4, only the LED light sources 181 shown with hatching can be turned on. In this case, the light emitted from the respective LED light source 181 takes an optical path as shown by a solid line in FIG. 4. That is, the illumination light generated in the LED light source 181 is condensed by the condenser lens 182 disposed corresponding to the LED light source 181, and then transmitted, in a state diffused by the diffusion plate 183, through the transparent plate 102 and the vessel bottom portion 512 of the culture vessel 510 upwardly from below. The illumination light is reflected on the inner surface of the vessel upper portion 511 of the culture vessel 510 and irradiates the cells 524 from obliquely above. Of the illumination light irradiating the cells 524, the light that has transmitted through the cells 524 passes through the vessel bottom portion 512 of the culture vessel 510 and the transparent plate 102 downwardly from above, and enters the objective lens 191. At this time, the light is refracted or scattered according to the shape and refractive index of the cells 524, or is attenuated according to the transmittance of the cells 524. The transmitted light corresponding to the cells 524 is collected by the objective lens 191 and imaged by the image sensor 153.

In the illumination unit 155, by changing over the LED light sources 181 for emitting the illumination light, the angle of the illumination light can be switched as shown by broken lines. By turning on only the LED light source 181 at a specific position in the circumferential direction of the objective lens 191, the cells 524 can be illuminated only from a specific circumferential direction. In addition, by turning on a plurality of LED light sources 181 arranged axially symmetrically with respect to the optical axis of the objective lens 191, illumination light with reduced illumination unevenness can be applied to the cells 524.

The illumination light from the illumination unit 155 is emitted from radially outside of the objective lens 191 and is reflected on the inner surface of the vessel upper portion 511 of the culture vessel 510, so that the illumination light irradiates the cells 524 from obliquely above and is collected, by the objective lens 191. Accordingly, by properly setting the angle of incidence on the cells 524, a contrast can be formed in the image of the cells 524. As a result, an easy-to-see image can be obtained even for a transparent subject such as a cell.

When the angle of incidence is smaller than the capture angle of the objective lens 191, the illumination is a bright-field illumination with less illumination unevenness. When the angle of incidence is larger than the capture angle of the objective lens 191, the illumination is a dark-field illumination in which the fine structures are emphasized. Further, when the angle of incidence is equal to the capture angle of the objective lens 191, the illumination is an oblique illumination in which the cells 524 are seen three-dimensionally.

Although FIG. 5 shows an example in which the red light sources 181R, the green light sources 181G, and the blue light sources 181B are arranged in a mixed manner, this arrangement is only an example and any arrangement is acceptable.

Explanation of the configuration of the observation system 1 continues by referring back to FIGS. 1 and 2. The observation apparatus 100 includes a driving mechanism 160. The driving mechanism 160 includes an X-axis driving mechanism 162 including, for example, a feed screw and an actuator for moving the support unit 165 in the X-axis direction, and a Y-axis driving mechanism 164 including, for example, a feed screw and an actuator for moving the support unit 165 in the Y-axis direction. Thus, the X-axis driving mechanism 162 and the Y-axis driving mechanism 164 function as a driving mechanism for moving the light receiving unit 151 in a direction perpendicular to the optical axis of the imaging optical system 152. As described above, the imaging position in the direction of the optical axis (Z-axis direction) of the imaging optical system 152 is changed by changing the position of the focus lens of the imaging optical system 152. Note that, instead of or in addition to the focus lens, the driving mechanism 160 may include a focus position driving mechanism including a Z feed screw and a Z actuator for moving the support unit 165 in the Z-axis direction.

The observation apparatus 100 may be provided with a sample driving mechanism for moving the sample 500 in the X-axis direction and the Y-axis direction instead of or in conjunction with the X-axis driving mechanism 162 and the Y-axis driving mechanism 164. In addition, the observation apparatus 100 may be provided with a sample driving mechanism for moving the sample 500 in the Z-axis direction instead of or in conjunction with the focus lens or the focus position driving mechanism.

The observation apparatus 100 repeatedly performs imaging using the light receiving unit 151 while changing the position of the image acquisition unit 150 in the X direction and the Y direction using the driving mechanism 160, and acquires a plurality of images at different positions. The observation apparatus 100 may combine these images to generate an image representing a single broad range.

Furthermore, the observation apparatus 100 may repeatedly perform imaging while changing the imaging position in the Z-axis direction and similarly changing the position in the X direction and the Y direction, combine the images, and sequentially acquire images at the respective Z-direction positions. In this way, the images of each part may be three-dimensionally obtained.

The observation apparatus 100 further includes a second control circuit 110, an image processing circuit 120, a second storage circuit 130, and a second communication device 140. The second communication device 140 is a communication device for performing communication with the control apparatus 300.

The second storage circuit 130 stores, for example, programs and various control parameters used in each unit of the observation apparatus 100, movement patterns of the image acquisition unit 150, and the like. Further, the second storage circuit 130 stores data and the like obtained by the observation apparatus 100.

The image processing circuit 120 performs various image processing on the image data obtained by the light receiving unit 151. The data after the image processing by the image processing circuit 120, for example, is stored in the second storage circuit 130 or transmitted to the control apparatus 300. Further, the image processing circuit 120 may perform various analyses based on the obtained images. For example, the image processing circuit 120 may extract an image of a cell or a cell group included in the sample 500, calculate the number of cells or cell groups, specify the pH value of the culture medium 522, and calculate the proliferation capability based on the number of cells and the pH value. The analysis result thus obtained is also stored in the second storage circuit 130 or transmitted to the control apparatus 300, for example.

The second control circuit 110 controls the operation of each unit included in the observation apparatus 100. The second control circuit 110 controls the operation of the driving mechanism 160 to control the position of the image acquisition unit 150, controls the imaging operation of the light receiving unit 151, controls the operation of the illumination unit 155, manages communication with the control apparatus 300 via the second communication device 140, and controls storing of data obtained by the observation apparatus 100.

The first control circuit 310, the second control circuit 110, and the image processing circuit 120 each include an integrated circuit such as a Central Processing Unit (CPU), an Application Specific Integrated Circuit (ASIC), a Field Programmable Gate Array (FPGA), or a Graphics Processing Unit (GPU). Each of the first control circuit 310, the second control circuit 110, and the image processing circuit 120 may be configured by a single integrated circuit or the like, or may be configured by combining a plurality of integrated circuits or the like. Further, the second control circuit 110 and the image processing circuit 120 may together be configured by one integrated circuit or the like. The operations of these integrated circuits can be performed in accordance with programs stored in, for example, the first storage circuit 330, the second storage circuit 130, or the integrated circuits. The first storage circuit 330 and the second storage circuit 130 may include one or more of a non-volatile memory such as a flash memory, and a volatile memory such as a static random access memory (SRAM) or a dynamic random access memory (DRAM).

### Functional Configuration of Observation System

The operation control of the observation apparatus 100 may be performed by the first control circuit 310 of the control apparatus 300, or the control apparatus 300 only send commands and the second control circuit 110 of the observation apparatus 100 may perform the control. In addition, the image processing may be performed by the image processing circuit 120 of the observation apparatus 100, or may be performed by the first control circuit 310 of the control apparatus 300 and/or the second control circuit 110 of the observation apparatus 100. In addition, for example, the first control circuit 310 of the control apparatus 300 or the second control circuit 110 of the observation apparatus 100 may perform analysis such as calculating the number of cells, calculating the pH value, and calculating the proliferation capability of the cells. Further, a dedicated analysis circuit for analysis may be provided in the control apparatus 300 or the observation apparatus 100.

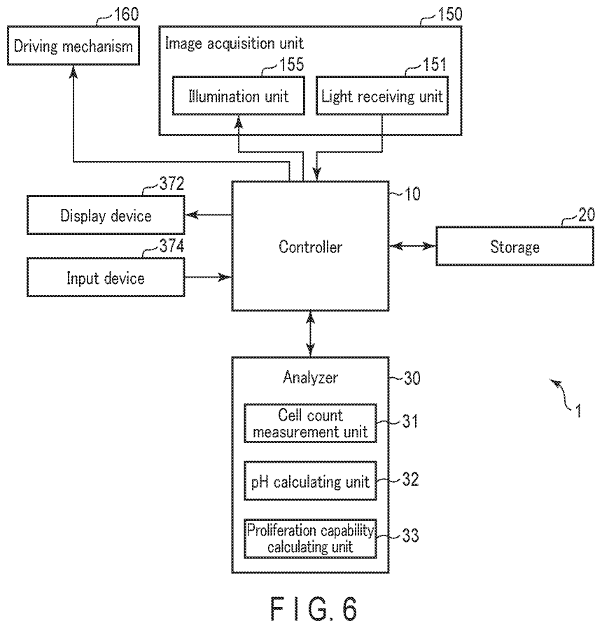

As described above, since various controls and various analyses may be performed by either the control apparatus 300 or the observation apparatus 100, the entire functional configuration of the observation system 1 is as shown in FIG. 6. That is, the observation system 1 includes an image acquisition unit 150 including the illumination unit 155 and the light receiving unit 151, and a controller 10 for controlling the operation of the driving mechanism 160 or the like. The controller 10 controls, for example, the display of the display device 372 in order to present various information to the user, and acquires an input related to the operation of the observation system 1 by the user for example, the input device 374. The controller 10 causes an analyzer 30 to analyze the image and the like acquired by the light receiving unit 151, and causes a storage 20 to store the acquired image, the analysis result, etc. The storage 20 may be implemented by the first storage circuit 330 of the control apparatus 300 or the second storage circuit 130 of the observation apparatus 100.

The analyzer 30 includes, for example, a function as a cell count measuring unit 31 or measuring the number of cells, a function as a pH calculating unit 32 for calculating the pH value of the culture medium, and a function as a proliferation capability calculating unit 33 for calculating the proliferation capability of cells based on the number of cells and the pH value.

### Observation of Observation System

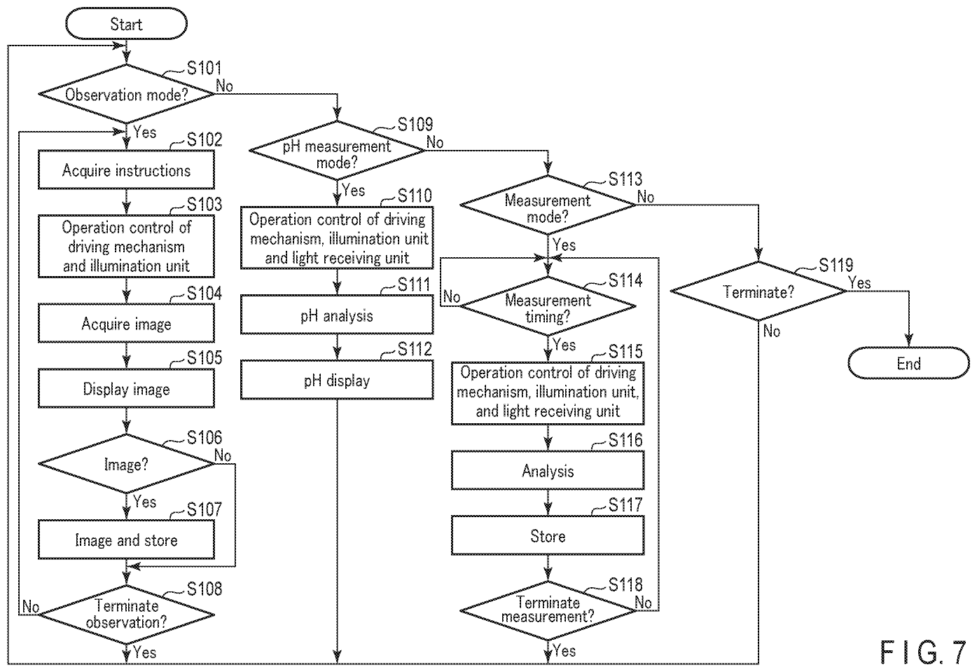

One example of an operation of the observation system 1 will be described with reference to the flowchart shown in FIG. 7. This process is started, for example, after the observation apparatus 100 is installed in the incubator 400, and the sample 500 is placed on the transparent plate 102 of the observation apparatus 100.

Here, the example where the observation system 1 has an observation mode, a pH measurement mode, and a measurement mode as its operation modes will be described. The observation mode is a mode in which the user operates the driving mechanism 160 and the image acquisition unit 150 using the input device 374 to display an image of a desired position of the sample 500 on the display device 372. The pH measurement mode is a mode in which the observation system 1 acquires a color image of the culture medium 522 of the sample 500 and acquires the pH value of the culture medium 522 based on the color of the culture medium 522. The measurement mode is a mode in which the observation system 1 acquires and analyzes an image of a predetermined position at a preset timing In the measurement mode, an image of the cells is acquired, and the number of cells, the pH value of the culture medium, the proliferation capability of the cells, and the like are calculated based on the image. The observation system 1 may have only some of these modes, or may further have other modes. The selection of the mode by the user can be performed at any timing by using, for example, the input device 374.

In step S101, the controller 10 determines whether or not the observation mode has been selected. When the observation mode has been selected, the process proceeds to step S102. In step S102, the controller 10 acquires, from the input device 374, information related to an operation instruction input by the user. In step S103, the controller 10 controls the operations of the driving mechanism 160 and the illumination unit 155 based on the acquired user's instructions. That is, the controller 10 causes the driving mechanism 160 to move the position of the image acquisition unit 150 according to the user's instructions. In the observation mode, the illumination unit 155 illuminates the sample 500, and the light receiving unit 151 acquires an image of the sample 500. Therefore, the controller 10 controls turning on/off, brightness, and the like of the illumination unit 155 according to user's instructions.

In step S104, the controller 10 causes the light receiving unit 151 to perform an imaging operation, and acquires image data obtained from the light receiving unit 151. In step S105, the controller 10 causes the display device 372 to display an image based on the obtained image data as a live view image. The user can observe the state of the sample 500 at a desired position by adjusting the position of the image acquisition unit 150 while viewing the image displayed on the display device 372. While viewing the image displayed on the display device 372, the user can change the focus position of the imaging optical system 152 or the position of the driving mechanism 160 in the Z-axis direction so as to focus on the cells 524 in the sample 500. A so-called auto focus function performed by the controller 10 based on an image may be used for focusing on the cell 524.

In the observation mode according to the present embodiment, the observation system 1 can acquire a high-duality image and store it in the storage 20 when the user desires. In step S106, the controller 10 determines whether or not an imaging instruction has been received from the user. If the instruction for imaging has not been received, the process proceeds to step S108. On the other hand, when the imaging instruction is received, the process proceeds to step S107. In step S107, the controller 10 causes the light receiving unit 151 to perform imaging in which a high-quality image is obtained, and stores the obtained image in the storage 20. Thereafter, the process proceeds to step S108.

In step S108, the controller 10 determines whether or not to end the observation mode. For example, the observation mode may be terminated based on a user's instruction. If the observation mode is not to be terminated, the process returns to step S102, and the processes of steps S102 to S107 described above are repeated. On the other hand, when the observation mode is terminated, the process returns to step S101.

If it is determined in step S101 that the observation mode has not been selected, the process proceeds to step S109. In step S109, the controller 10 determines whether or not the pH measurement mode has been selected. When the pH measurement mode has been selected, the process proceeds to step S110.

In step S110, the controller 10 controls the operations of the driving mechanism 160, the illumination unit 155, and the light receiving unit 151. That is, the driving mechanism 160 moves the image acquisition unit 150 to a position where the culture medium 522 of the sample 500 can be imaged. The focus position of the imaging optical system 152 is aligned with the region of the culture medium 522, not the cells 524. The control unit 10 synchronizes the illumination by the illumination unit 155 and the imaging by the light receiving unit 151 to acquire a color image. More specifically, as the illumination unit 155 is configured to sequentially perform illumination light emissions of three colors and the light receiving unit 151 can acquire a single color image, the following operation is performed. The controller 10 causes the illumination unit 155 to sequentially perform illumination light emissions of three colors, and causes the light receiving unit 151 to image the culture medium 522 while the illumination light emission of the respective color is performed. In this way, the controller 10 can acquire images of the respective colors for the culture medium 522.

The above description has assumed that, in order to image the culture medium, the image acquisition unit 150 is moved by the driving mechanism 160, and the focus position of the imaging optical system 152 is aligned with the region of the culture medium 522, not the cells 524. However, the embodiment may adopt any operation as long as it allows for the imaging of a part where the cells are not present. Therefore, the movement of the focus position may be omitted, and only the image acquisition unit 150 may be moved to a location where no cells are present using the driving mechanism 160. Alternatively, only the focus position may be changed without moving the image acquisition unit 150 using the driving mechanism 160.

In step S111, the controller 10 performs an analysis for specifying the pH value of the culture medium 522 based on the obtained image of each color or the color image related to the culture medium 522. For example, the color of the culture medium with phenol red added changes according to the pH value. The pH value of the culture medium can be specified based on this color.

For example, Jpn. Pat. Appln. KOKAI Publication No. S62-115257 discloses that the following relationship is established. That is, assuming that the absorbance values at wavelengths of 430 nm, 558 nm, and 630 nm are A₄₃₀, A₅₅₈ and A₆₃₀, respectively, pH₀, pH₁₀, and pH₂₀, which are the pH values of the respective Dulbecco MEM media each containing 0.001% phenol red and respectively having fetal bovine serum concentrations of 0%, 10%, and 20%, are represented by the following equations:

$${pH}_{0} = log ( \frac{{A}_{430} - {A}_{630}}{{A}_{558} - {A}_{630}} ) \times 1.08 - 7.31$$

$${pH}_{10} = log ( \frac{{A}_{430} - {A}_{630}}{{A}_{558} - {A}_{630}} ) \times 1.15 - 7.38$$

$${pH}_{20} = log ( \frac{{A}_{430} - {A}_{630}}{{A}_{558} - {A}_{630}} ) \times 1.27 - 7.47$$

Similarly, assuming that the absorbance values at wavelengths of 441 nm, 578 nm, and 634 nm are A₄₄₁, A₅₇₈, and A₆₃₄, respectively, the pH value of the Dulbecco MEM culture medium containing 0.001% phenol red and 10% fetal bovine serum is expressed by the following equation:

$$pH = log ( \frac{{A}_{441} - {A}_{634}}{{A}_{578} - {A}_{634}} ) \times 1.19 - 7.86$$

It has been shown that such a relationship can be equally obtained from performing measurement using a filter having a half-value width and from performing measurement using light of a single wavelength.

Also, in the analysis of step S111 of the present embodiment, the pH value of the culture medium 522 can be specified using the acquired image with reference to the above-described relationship determined according to various conditions, including, for example, the configuration of the observation system 1 such as the wavelength for the obtained image, the condition of the sample 500, and the like.

Although an example in which the absorbance is measured for three wavelengths to obtain the pH value is shown here, the pH can be calculated by measuring the absorbance for two or more wavelengths.

Based on the image of each color (each wavelength), the transmittance of the culture medium 522 for the light of each color is obtained. Here, the intensity of light for each color (for each wavelength) that has not passed through the sample 500, which will be used as a reference intensity for calculating the transmittance, may be obtained in advance of a series of observations while, for example, an empty culture vessel containing no culture medium is disposed. If the observations in the incubator 400 are planned, the reference intensity is also preferably acquired in the incubator 400. The reference intensity obtained in advance is stored, for example, in the recording unit 20. By comparing such a reference intensity with an image obtained by imaging the sample 500, the transmittance for each color (each wavelength) can be obtained.

In addition, the reference intensity is not limited to the use for obtaining the transmittance when measuring the pH value, and may be used, for example, when correcting the color information of the obtained image in comparison with the transmittance and color data prepared in advance in the image analysis.

In step S112, the controller 10 causes the display device 372 to display the obtained pH value. By utilizing the pH measurement mode as described above, the user can know the pH value of the culture medium 522 of the sample 500. Thereafter, the process returns to step S101, and the above-described process is repeated.

Note that the pH measurement mode may be executed based on a user's instructions, or may be repeatedly executed over time according to a predetermined schedule. Such execution over time allows monitoring a change of the pH value over time. The obtained pH value data can be stored in the storage 20.

If it is determined in step S109 that the pH measurement mode has not been selected, the process proceeds step S113. In step S113, the controller 10 determines whether or not the measurement mode has been selected. When the measurement mode has been selected, the process proceeds to step S114.

In step S114, the controller 10 determines whether or not it is time to perform measurement. In the present embodiment, the observation system 1 can repeatedly acquire an image at a predetermined time interval, for example, every hour, or at a predetermined timing, and perform analysis based on the image. In this way, the observation system 1 may, for example, obtain time-lapse images of the sample 500 in a predetermined range. If it is not the time to perform the measurement, the process repeats step S114 as a standby state. On the other hand, if it is time to perform the measurement, the process proceeds to step S115.

In step S115, the controller 10 controls the operations of the driving mechanism 160, the illumination unit 155, and the light receiving unit 151 to image a predetermined position of the sample 500 under predetermined conditions. For example, when it is set that an image of an area of the sample 500 is to be acquired, the controller 10 controls the driving mechanism 160 to move the position of the image acquisition unit 150 within the area so that the images of this area are sequentially taken by the light receiving unit 151. At this time, the focus position of the imaging optical system 152 is adjusted so as to focus on the cells 524. In addition, when the pH value of the culture medium is measured together with the imaging of the cells or instead of the imaging of the cells, the focus position of the imaging optical system 152 is adjusted so as to focus on the culture medium 522 at a height where it does not have the cells 524, and the imaging is performed. In the pH value measurement, by adjusting the focus position to focus on the culture medium 522 at the height where it does not have the cells 524, it is possible to prevent an error occurrence in the measured transmitted light intensity due to a difference in the state or number of the cells 524.

In step S116, the controller 10 causes the analyzer 30 to perform a predetermined analysis or the like based on the image(s) obtained by imaging. The analyzer 30 synthesizes a plurality of obtained images of the cells to, for example, create one synthesized. image indicating a state of a predetermined area. The analyzer 30, for example, counts the number of cells and the like and specifies the size of a colony based on the obtained image. The analyzer 30, for example, calculates the pH value of the culture medium based on the obtained image of the culture medium. The analyzer 30 can also calculate the proliferation capability of the cells in culture. This proliferation capability may be expressed by, for example, ΔpH/CN using a change amount ΔpH of the pH value with elapsed time and a calculated cell number CN. The pH value of the culture medium changes with cellular metabolism. Thus, ΔpH per unit cell can be used as an indicator of cell activity. In step S113, the controller 10 stores the obtained image(s) and the analysis result in the storage 20.

In step S118, the controller 10 determines whether or not to terminate the measurement mode. For example, when a predetermined series of image acquisition and analysis is completed, it is determined to terminate the measurement. If the measurement mode is not to be ended, the process returns to step S114, and the processes of steps S114 to S117 described above are repeated. On the other hand, when ending the measurement mode, the process returns to step S101.

If it is determined in step S113 that the measurement mode has not been selected, the process proceeds to step S119. In step S119, the controller 10 determines whether or not to terminate the processing. For example, when the user inputs an end of using the observation system 1, the processing is determined to be terminated. If it is determined that the processing is not to be terminated, the process returns to step S101, and the above-described processing is repeated. When it is determined that termination should occur, the series of processing is finished.

According to the observation system 1 of the present embodiment as described above, the sample 500 left still in the incubator 400 can be observed, recorded, and subjected to various analyses as it is, and the pH value of the culture medium can also be measured as it is. The observation apparatus 100 can measure the pH value without touching the culture medium. In this way, the observation system 1 can measure the pH value of the culture medium while preventing contamination. Further, when the observation system 1 is used, various information can be obtained by numerical values, so that the user can make an objective judgment as compared with the cases where various judgments must be made by visual observation.

In the observation apparatus 100 of the observation system 1, the illumination light is emitted from the illumination unit 155 provided at the image acquisition unit 150 in the housing 101, and the culture medium 522 or the cells 524 are illuminated by the light reflected at the upper portion 511 of the culture vessel 510 of the sample 500. This simplifies the configuration of the observation apparatus 100. Further, no special vessel is required for the sample 500, and the observation apparatus 100 can be used regardless of the shape of the culture vessel 510.

In the observation apparatus 100 according to the present embodiment, the illumination unit 155 including the illumination optical system 156, and the imaging optical system 152 including the objective lens 191 are disposed below the cells 524. Therefore, unlike in the structure of the conventional observation apparatuses, which use transmitted light and in which the illumination optical system 156 and the imaging optical system 152 would be disposed with the sample 500 interposed therebetween, the illumination unit 155 and the light receiving unit 151 can be arranged together on only one side of the cells 524. As a result, the thickness of the observation apparatus 100 can be reduced. Even when such a thin observation apparatus 100 is used, the user can observe a subject such as cells or the like, without labeling it by imaging with transmitted light.

The observation system 1 can obtain various information including an image of the cells, the number of cells, and the pH value of the culture medium even when using the observation apparatus 100 having such a simple configuration. Since both the observation of the cells and the measurement of the pH value of the culture medium can be performed by using the light receiving unit 151 and the illumination unit 155, the configuration of the observation apparatus 100 can be simplified as compared with the cases of having separate configurations for observation and pH measurement.

The outer shape of the observation apparatus 100 may be, for example, a rectangular parallelepiped. The observation apparatus 100 having a simple shape with little unevenness, such as a rectangular parallelepiped, has an advantage that cleaning including disinfection can be easily done when it is applied to use in a cell culture in which contamination or the like is a problem. In addition, the observation apparatus 100 having such a shape is also convenient for placing into and taking out of the incubator 400. Further, the shape of the observation apparatus 100 having no structure above the transparent plate 102 contributes to space saving, thus is advantageous in installation in the incubator 400 having a relatively limited space. Further, the place to install the observation apparatus 100 is not limited to the incubator 400, and it can be installed in, for example, a clean bench, and even in such a case, the simple shape of the observation apparatus 100 is effective in reserving a wide working space above the transparent plate 102.

In the observation apparatus 100 according to the present embodiment, the image acquisition unit 150 including the illumination unit 155 and the light receiving unit 151 is moved by the driving mechanism 160. In general, it is preferable not to apply an unnecessary shock to the cells in a culture. For example, when cells are observed with a general microscope, a sample is taken out from an incubator and placed in the microscope, or the sample is moved on the stage of the microscope. On the other hand, according to the observation apparatus 100, a wide range of the sample 500 can be observed in a state where the sample 500 in a culture is left stationary. Thus, the observation system 1 allows various observations or measurements to be made without straining the cells.

### First Modification of the First Embodiment

A first modification of the first embodiment will be described. Here, the difference from the first embodiment will be explained and the explanation of similar parts will be omitted. In this modification, the illumination unit 155 and the light receiving unit 151 have different configurations. In the first embodiment, the image sensor 153 is a monochromatic sensor, and the light source 157 individually emits red light, green light, and blue light. In the first embodiment, a color image is acquired in a time-division manner by synchronizing the imaging by the light receiving unit 151 and the illumination by the illumination unit 155.

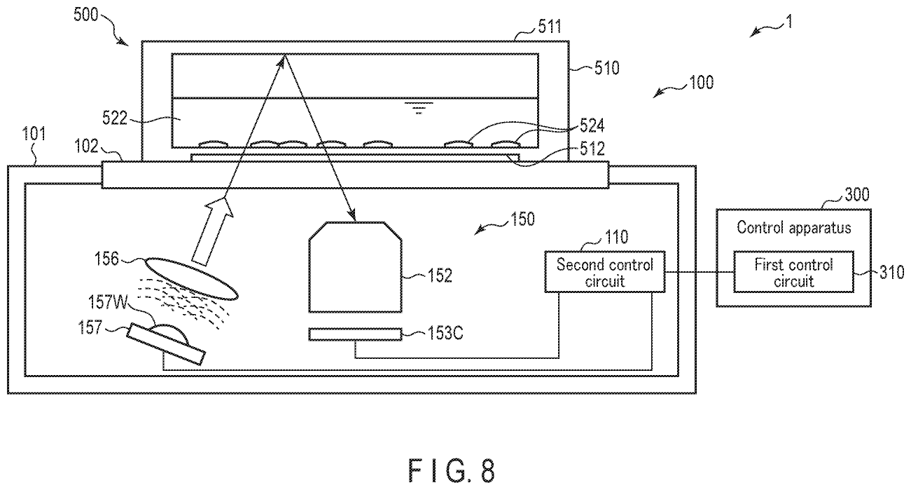

In contrast, in the present modification, the light source 157 includes, for example, one light source that emits illumination light including a plurality of colors. The light source may be, for example, a white light source 157W that emits white illumination light as shown in FIG. 8. In this modification, the image sensor 153 is a color sensor 153C. The color sensor 153C is a sensor in which, for example, red filters, green filters, and blue filters as optical elements are provided on the image sensor. The color sensor 153C can detect the intensity of light for each color by color-separating the light included in the white light. A color image may also be acquired by a combination of the white light source 157W and the color sensor 153C. Based on the color image, analysis of the pH value of the culture medium and so on can be performed.

The configuration of the light source 157 may include a plurality of monochromatic light sources as in the first embodiment, and the configuration of the image sensor may be the color sensor 153C. In this case, even if the red light source 157R, the green light source 157G, and the blue light source 157B included in the light source 157 emit the illumination light at the same time, a color image can be obtained by color separation by the color sensor 153C.

### Second Modification of the First Embodiment

A second modification of the first embodiment will be described. Here, the difference from the first embodiment will be explained and the explanation of the similar parts will be omitted. In the aforementioned first embodiment, the observation apparatus 100 is configured to be able to acquire an image of the sample 500 and the pH value of the culture medium 522 of the sample 500. On the other hand, in the present modification, the observation apparatus 100 is configured so as to be able to acquire a pH value, while not acquiring an image. The light receiving unit 151 includes an optical sensor for detecting light intensity as a light receiving device instead of the image sensor 153. Other configurations of the observation apparatus 100 are the same as those of the first embodiment.

For example, the illumination light is emitted to the sample 500 on the transparent plate 102 from the illumination unit 155 provided in the housing 101. The illumination light enters the culture vessel 510 from below, is reflected by the upper portion of the culture vessel 510, and is incident on the light receiving unit 151 provided in the housing 101. The optical sensor in the light receiving unit 151 detects, for each color of the light, the light intensity corresponding to the color of the culture medium 522 through which the incident light has passed. Based on the detected intensity of the light of each color, the observation system 1 can obtain the pH value of the culture medium 522.

In the instances where only the pH value of the culture medium is required, the apparatus can be simplified by adopting an apparatus configuration in which only the culture medium is observed as in the present modification.

### Second Embodiment

A second embodiment will be described. Here, the difference from the first embodiment will be described, and the same reference numerals are assigned to the same parts and the description thereof will be omitted. In the first embodiment, the illumination unit 155 is disposed in the image acquisition unit 150 provided in the housing 101 of the observation apparatus 100. On the other hand, according to the second embodiment, the image acquisition unit 150 is not provided with the illumination unit 155, and instead, an external illumination unit 255 is provided outside the housing 101 and at a position facing the image acquisition unit 150 with the sample 500 interposed therebetween.

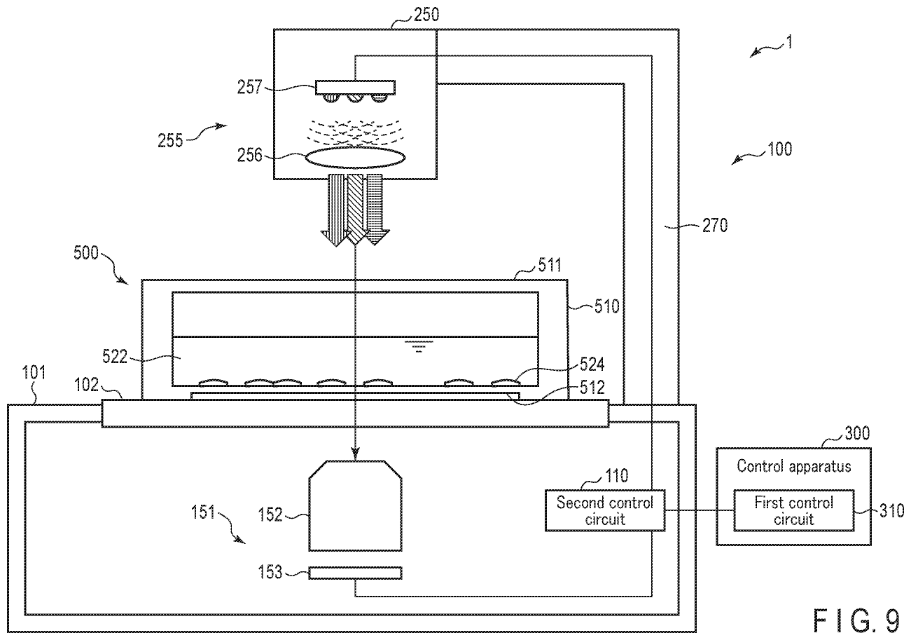

FIG. 9 schematically shows a configuration example of the observation system 1 according to the present embodiment. In FIG. 9, the driving mechanism 160 and the like are omitted as in FIG. 3. As shown in FIG. 9, the light receiving unit 151 including the imaging optical system 152 and the image sensor 153 is provided in the housing 101 of the observation apparatus 100. On the other hand, the external illumination unit 255 for emitting the illumination light is disposed oppositely to the light receiving unit 151 with the transparent plate 102 on the outside of the housing 101 interposed therebetween.

The external illumination unit 255 includes an external light source 257 and an external illumination optical system 256 provided in an illumination supporting unit 250. The external light source 257 can individually emit red light, green light, and blue light as illumination light, similarly to the light source 157 according to the first embodiment. The external light source 257 includes, for example, an LED or the like. The illumination light from the external light source 257 is emitted from the external illumination unit 255 via the external illumination optical system 256 to illuminate the sample 500.

Since the external illumination unit 255 is disposed at a position opposed to the light receiving unit 151 with respect to the sample 500, the illumination light emitted from the external illumination unit 255 passes through the sample 500 and reaches the light receiving unit 151. That is, the light receiving unit 151 images the sample 500 that was illuminated by the transmitted light.

As shown in FIG. 9, the illumination support unit 250 of the external illumination unit 255 is supported by, for example, a support column 270. The support column 270 may be fixed to the housing 101. The support column 270 may be installed in the incubator 400 independent of the housing 101. The illumination support unit 250 does not need to be fixed to the support unit 270, and may be furnished on, for example, a ceiling or a wall of the incubator 400. The illumination support unit 250 may be configured to be placed on the culture vessel 510 of the sample 500.

The external illumination unit 255 is configured such a manner that its illumination condition does not change even if the position of the light receiving unit 151 is changed by the driving mechanism 160. For example, the external illumination unit 255 may be located sufficiently far from the transparent plate 102 in order to uniformly illuminate any position of the sample 500 disposed on the transparent plate 102. The external illumination unit 255 may be configured as a sheet-like illumination apparatus so as to uniformly illuminate a wide area.

The external illumination unit 255 may move in accordance with the movement of the light receiving unit 151. That is, the illumination support unit 250 may be provided with a driving mechanism for moving the external illumination unit 255 in the X-axis direction and the Y-axis direction. The driving mechanism may operate in synchronization with the driving mechanism 160 in the housing 101 for moving the light receiving unit 151, and may operate so that the external illumination unit 255 and the light receiving unit 151 always face each other. Further, the observation apparatus 100 may omit the driving mechanism 160 in the housing 101 for moving the light receiving unit 151, and may be fixed at a position so that the light receiving unit 151 and the external illumination unit 255 of the observation apparatus 100 face each other. In this case, in order to image a wide range of the sample 500, the observation apparatus 100 may include a sample driving mechanism for moving the sample 500 in the X-axis direction and the Y-axis direction.

According to the observation system 1 of the second embodiment, the sample 500 left still in the incubator 400 can be observed, recorded, and subjected to various analyses as it is, and the pH value of the culture medium can also be measured as it is. According to the present embodiment as well, a special vessel is not required for the sample 500, and the observation system 1 can be used regardless of the shape of the culture vessel 510. Since the observation apparatus 100 can perform both the observation of the cells and the measurement of the pH value of the culture medium by using the light receiving unit 151 and the external illumination unit 255. the configuration of the observation apparatus 100 can be simplified as compared with the cases of having separate configurations for observation and pH measurement.

The above-described embodiments and modifications thereof can be appropriately combined. For example, in the second embodiment, a white light source and a color sensor can be used as in the first modification of the first embodiment. In the second modification of the first embodiment, a white light source can be used as in the first modification of the first embodiment.

Among the techniques described in the embodiments, the control mainly described in the flowchart can be realized using a program. This program can be stored in a storage medium or the storage. There are various methods of storing the program in the storage medium or the storage, and the storing may be performed at the time product shipment, may be performed using a distributed storage medium, or may be formed using downloading via the Internet. Further, all or a part of the above-described processing may be, for example, performed using artificial intelligence, etc. constructed using deep learning.

Although a monochromatic light source and an LED have been described as the light source 157, it is sufficient if the light source 157 can perform three-color light emissions, and it may also include a multi-color light source and a monochromatic light source. Additional advantages and modifications will readily occur to those skilled in the art. Therefore, the invention in its broader aspects is not limited to the specific details, representative devices, and illustrated examples shown and described herein. Accordingly, various modifications may be made without departing from the spirit or scope of the general inventive concept as defined by the appended claims and their equivalents.

What is claimed is:

1. An observation system comprising:
an illumination unit including a light source from which emits an illumination light including light of a plurality of colors, and configured to emit the illumination light for a culture vessel so that the illumination light travels from a first side to a second side of the culture vessel, the culture vessel being configured to store a culture medium for culturing a cell, and the first side and the second side being defined by interposing the culture medium; and
a light receiving unit including an optical sensor which acquires a light intensity, and configured to receive, on the first side, light emitted from the illumination unit, entering the culture vessel from the first side, reflected at the second side, and transmitted through the culture medium, and to acquire a light intensity for each color in the received light, the light intensity being used for calculation of a pH value of the culture medium.

2. The observation system according to claim 1, wherein
the optical sensor includes an image sensor which acquires a light intensity and an image,
the image sensor is configured to:
receive the light of the plurality of colors transmitted through the culture medium, and
acquire an image of the cell and the light intensity for each color in the received light for calculation of the pH value of the culture medium.

3. The observation system according to claim 2, wherein
the light source includes a plurality of monochromatic light sources configured to independently emit a respective one of the light of the plurality of colors, and
the light receiving unit further includes an optical device, and is configured to acquire the light intensity for each color by color separating the received light using the optical device.

4. The observation system according to claim 2, wherein
the optical sensor includes a sensor which receives light as a monochromatic color,
the light source includes a plurality of monochromatic light sources configured to independently emit each of the light of the plurality of colors in a time-division manner, and
the optical sensor is configured to acquire the light intensity for each color in a time-division manner.

5. The observation system according to claim 2, wherein
the light source includes a single light source from which emits the illumination light, and
the light receiving unit further includes an optical device, and is configured to acquire the light intensity for each color by color separating the received light using the optical device.

6. The observation system according to claim 2, wherein
the light receiving unit further includes an optical system having an optical axis, and
the observation system further comprises an actuator configured to move the light receiving unit in a direction perpendicular to the optical axis of the optical system in the light receiving unit.

7. The observation system according to claim 2, wherein
the light receiving unit further includes an optical system having an optical axis, and
the observation system further comprises an actuator configured to move the culture vessel in a direction perpendicular to the optical axis of the optical system in the light receiving unit.

8. The observation system according to claim 2, wherein
the light receiving unit further includes an optical system having an optical axis, and
the observation system further comprises an actuator configured to move a focus position of the light receiving unit in a direction of the optical axis of the optical system in the light receiving unit.

9. The observation system according to claim 2, wherein
the light receiving unit further includes an optical system having an optical axis, and
the observation system further comprises an actuator configured to move the culture vessel in a direction of the optical axis of the optical system in the light receiving unit.

10. The observation system according to claim 8, wherein the actuator is configured to
adjust the focus position of the light receiving unit to a surface of the cell for an imaging to acquire the image of the cell, and
adjust the focus position of the light receiving unit to a surface of the culture medium for an imaging to calculate the pH value of the culture medium.

11. The observation system according to claim 9, wherein the actuator is configured to
adjust a position of the culture vessel so that a focus position of the light receiving unit to a surface of the cell for an imaging to acquire the image of the cell, and
adjust the position of the culture vessel so that the focus position of the light receiving unit to a surface of the culture medium for an imaging to calculate the pH value of the culture medium.

12. The observation system according to claim 2, further comprising:
a control circuit configured to calculate a cell number based on the image of the cell.

13. The observation system according to claim 1, wherein
the illumination unit further includes an optical system,
the light receiving unit further includes an optical system, and
the optical system of at least one of the illumination unit and the light receiving unit includes an optical system for a single wavelength.

14. The observation system according to claim 1, wherein
the illumination unit further includes an optical system,
the light receiving unit further includes an optical system, and
the optical system of at least one of the illumination unit and the light receiving unit includes an optical system for color image detection.

15. The observation system according to claim 1, further comprising:
a housing with a sealed configuration, the housing comprising a horizontal, optically transparent plate at its top during installation, wherein
the illumination unit is disposed inside the housing to emit the light in a direction of the transparent plate, and
the light receiving unit is disposed inside the housing to receive the light from the direction of the transparent plate.

16. The observation system according to claim 1, further comprising:
a control circuit configured to calculate the pH value of the culture medium based on the light intensity for each color.

17. The observation system according to claim 2, wherein the light intensity for calculating the pH value of the culture medium is acquired from an image of the culture medium acquired by the image sensor.

18. An observation method using an observation system including an illumination unit including a light source from which emits an illumination light including light of a plurality of colors, and configured to emit the illumination light for a culture vessel so that the illumination light travels from a first side to a second side of the culture vessel, the culture vessel being configured to store a culture medium for culturing a cell, and the first side and the second side being defined by interposing the culture medium and a light receiving unit including an optical sensor which acquires a light intensity, and configured to receive, on the first side, light emitted from the illumination unit, entering the culture vessel from the first side, reflected at the second side, and transmitted through the culture medium, and to acquire a light intensity for each color in the received light, the light intensity being used for calculation of a pH value of the culture medium, wherein the optical sensor includes an image sensor which acquires a light intensity and an image, the image sensor is configured to receive the light of the plurality of colors transmitted through the culture medium, and acquire an image of the cell and the light intensity for each color in the received light for calculation of the pH value of the culture medium, the method comprising:
acquiring an image of the cell by setting a focus position of the light receiving unit on the cell;
acquiring an image of the culture medium by setting the focus position of the light receiving unit on the culture medium; and
calculating a pH value of the culture medium based on the image of the culture medium.

19. A non-transitory computer-readable storage medium storing a program to control an observation system including an illumination unit including a light source from which emits an illumination light including light of a plurality of colors, and configured to emit the illumination light for a culture vessel so that the illumination light travels from a first side to a second side of the culture vessel, the culture vessel being configured to store a culture medium for culturing a cell, and the first side and the second side being defined by interposing the culture medium and a light receiving unit including an optical sensor which acquires a light intensity, and configured to receive, on the first side, light emitted from the illumination unit, entering the culture vessel from the first side, reflected at the second side, and transmitted through the culture medium, and to acquire a light intensity for each color in the received light, the light intensity being used for calculation of a pH value of the culture medium, wherein the optical sensor includes an image sensor which acquires a light intensity and an image, the image sensor is configured to receive the light of the plurality of colors transmitted through the culture medium, and acquire an image of the cell and the light intensity for each color in the received light for calculation of the pH value of the culture medium, and wherein the program causes a computer of the observation system to:
operate a process of acquiring an image of the cell by setting a focus position of the light receiving unit on the cell;
acquire an image of the culture medium by setting the focus position of the light receiving unit on the culture medium; and
calculate a pH value of the culture medium based on the image of the culture medium.
